(19) United States
(12) Patent Application Publication
(10) Pub. No.: US 2011/0054039 A1
(43) Pub. Date: Mar. 3, 2011

(54) USE OF ALKYL SULFATES OR SULFONATES AGAINST PROPIONIBACTERIUM ACNES

(76) Inventors: Elizabeth A. Eady, Bradford (GB); Andleeb Qureshi, Bradford (GB); Scott Seville, Bradford (GB)

(21) Appl. No.: 12/937,632

(22) PCT Filed: Apr. 16, 2009
(86) PCT No.: PCT/GB09/50370

§ 371 Date: Nov. 15, 2010

(30) Foreign Application Priority Data
Apr. 16, 2008 (GB) 0806851.2

(51) Int. Cl. A61K 31/10; A01N 41/10; A61P 17/10; A61P 31/04; A01P 15/00

(52) U.S. Cl. 514/711

(57) ABSTRACT

An alkyl sulphate or sulphonate having a carbon chain length of 11 or greater, for use in the treatment of a condition which is caused by, transmitted by and/or exacerbated by propionibacterial activity, in particular acne.

### FIELD OF THE INVENTION

This invention relates to the use of certain compounds as antibacterial and anti-acne agents.

### BACKGROUND TO THE INVENTION

Alkyl sulphates and sulphonates, in particular longer chain alkyl sulphates such as those derived from fatty acid sources, are known for use as anionic surfactants. Sodium tetradecyl sulphate (STS), for example, is used as a wetting agent in various industrial applications. Common synonyms for STS include sodium myristyl sulphate and 1-tetradecanol hydrogen sulphate sodium salt.

The alkyl chain of STS can be either linear, as in formula (I) below (CAS registration no. 1191-50-0):

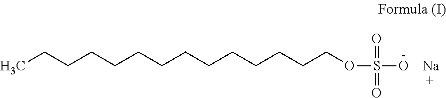

or branched, as in the compound of formula (II) below:

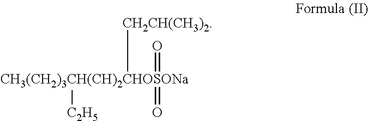

The chemical name for the compound of formula (II) is 7-ethyl-2-methyl-4-hendecanol sulphate sodium salt. It is commercially known as Sotradecol® (Cumberland Pharmaceuticals Inc, USA), which is a FDA-approved sterile non-pyrogenic sclerosing agent, and as Fibro-Vein™ (STD Pharmaceutical Products Ltd) which is a mixture of four stereoisomers.

It has now surprisingly been found that alkyl sulphates and sulphonates such as STS can be active against propionibacteria, the bacteria implicated in inflammatory acne.

### STATEMENTS OF THE INVENTION

According to a first aspect of the present invention there is provided an alkyl sulphate or sulphonate having a carbon chain length of 11 or greater, for use in the treatment of a condition affecting the human or animal body, which condition is caused by, transmitted by and/or exacerbated by (in particular either caused or transmitted by) propionibacterial activity. The condition may in particular be acne.

In the context of the present invention, treatment of a condition encompasses both therapeutic and prophylactic treatment, of either an infectious or a non-infectious condition, in either a human or animal but in particular a human. It may involve complete or partial eradication of the condition, removal or amelioration of associated symptoms, arresting subsequent development of the condition, and/or prevention of, or reduction of risk of, subsequent occurrence of the condition. It will typically involve use of the alkyl sulph(on)ate as an antibacterial agent, and/or as an anti-acne agent.

In an embodiment of the invention, the alkyl sulph(on)ate is for use against one or more strains of Propionibacterium acnes and/or in some instances P. granulosum.

Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment (which embraces prevention or reduction) of any of these symptoms, and references to use as an anti-acne agent may be construed accordingly. In particular, the treatment of acne encompasses the treatment (including prevention) of lesions and/or scarring associated with acne. It also encompasses the inhibition of propionibacterial activity which could cause or be otherwise associated with acne or its symptoms.

In general, the present invention will be used for the treatment of symptoms which are directly due to acne rather than for instance infections which may arise as a consequence of treating acne with other actives such as antibiotics, and/or secondary infections caused by opportunistic pathogens, which can arise in skin already affected by acne.

Instead or in addition, the alkyl sulph(on)ate may be for use against an opportunistic infection which is caused, transmitted and/or exacerbated by (in particular caused by) propionibacteria, for instance an infection associated with an indwelling surgical device (a prosthetic joint, for example). It may be for use in treating an infected wound, burn or ulcer. It may be for use against any other infection or condition which involves or can involve propionibacteria, in particular an eye infection such as endophthalmitis, or in cases body odour.

A second aspect of the present invention provides the use of an alkyl sulphate or sulphonate having a carbon chain length of 11 or greater, in the manufacture of a medicament (typically a formulation) for the treatment of a condition which is caused by, transmitted by and/or exacerbated by (in particular either caused or transmitted by) propionibacterial activity. The condition may be selected from those listed above in connection with the first aspect of the invention. It may in particular be acne. The alkyl sulph(on)ate will typically be used as an antibacterial agent, and/or as an anti-acne agent, in the manufacture of the medicament.

In an embodiment of the invention, the alkyl sulph(on)ate may have a carbon chain length of at least 12, or in cases of at least 13 or 14. It may have a maximum carbon chain length of 22, or 20, or 19 or 18 or 17 or 16 or in cases 15 or 14. In an embodiment, it is a C11 to C20 alkyl sulph(on)ate, or a C11 to C18 alkyl sulph(on)ate, or a C11 to C16 alkyl sulph(on)ate, or a C11 to C15 alkyl sulph(on)ate.

In particular where it is an alkyl sulphate, it may have a carbon chain length from 11 to 20, or from 11 to 18, or from 12 to 18 or 20, or from 13 to 18 or 20; it may for example be a C12 or C1-4 alkyl sulph(on)ate (which includes lauryl (dodecyl) and myristyl (tetradecyl) sulph(on)ates), more particularly a C1-4 alkyl sulph(on)ate. Other suitable alkyl sulph(on)ates, in particular sulphates, include tridecyl, cetyl (hexadecyl) and stearyl (octadecyl) sulph(on)ates.

In particular where the compound is an alkyl sulphonate, it may have a carbon chain length of at least 12 or 13; its chain length may for example be up to 18 or 16 or 15, ideally from 12 to 16 or from 12 or 15, or in cases from 13 to 15, or from 14 to 16, such as 14.

Generally speaking the alkyl chain may be either straight chain or branched. In cases it may include one or more unsaturated carbon-carbon bonds, for example as in palmitoleyl, elaidyl, oleyl and linoleyl groups. In cases however it may be preferred for the alkyl chain not to include any unsaturated carbon-carbon bonds. In cases it may be preferred for the alkyl chain to be linear rather than branched.

In the context of the present invention, the terms ‘alkyl sulphate’ and ‘alkyl sulphonate’ are not intended to embrace alkyl ether sulphates and alkyl ether sulphonates respectively.

A particularly preferred alkyl sulph(on)ate for use in the present invention is a C14 alkyl sulph(on)ate such as a linear n-tetradecyl sulph(on)ate or a branched chain isomer thereof. In an embodiment, the alkyl sulph(on)ate is an n-tetradecyl sulph(on)ate, in particular sodium n-tetradecyl sulph(on)ate. In another embodiment, the alkyl sulph(on)ate is a 7-ethyl-2-methyl-4-hendecanol sulph(on)ate, again in particular the sodium salt.

In an embodiment of the invention the alkyl sulph(on)ate is an alkyl sulphate. In an embodiment, it is an alkyl sulphonate.

An alkyl sulphate or sulphonate may be used in the form of a derivative, for example a salt with any suitable, preferably pharmaceutically acceptable, cation. Examples include metal cations (in particular alkali metals such as lithium, sodium or potassium and alkaline earth metals such as calcium and magnesium); and ammonium ions. Particularly preferred metal cations are sodium, potassium and magnesium, in particular sodium. Ammonium ions may be substituted ammonium ions, for example alkyl- or alkanolyl-substituted ammonium ions such as methyl-, dimethyl-, trimethyl-, tetramethyl-, ethyl-, diethyl or triethylammonium ions or ethanol-, diethanol- or in particular triethanolammonium ions, or other substituted (typically quaternary) ammonium ions such as dimethyl piperidinium cations, or simply NH₄⁺, of which the latter may be preferred. A mixture of two or more different cations may be present.

A ‘derivative’ of an alkyl sulphate or sulphonate may be a pharmaceutically acceptable (which term includes acceptable for veterinary use) derivative. It may be for example a salt, complex or solvate or a so-called ‘prodrug’ form or protected form which reverts to an active form of the relevant compound at an appropriate time on or after administration. It may be a free acid—for example an alkyl sulphonic acid—from which the sulphate or sulphonate can be derived. Preferably, however, the alkyl sulph(on)ate is present in the form of the sulph(on)ate alone.

A formulation prepared or used according to the invention may contain a mixture of two or more different alkyl sulphates or sulphonates or derivatives thereof.

In accordance with the invention, the alkyl sulph(on)ate is used as an antibacterially active agent. Instead or in addition, it may be used as an anti-acne agent (ie as an agent which is active against acne (which includes against a symptom and/or a cause of acne) and/or against one or more micro-organisms associated with acne). Suitably it is not used purely or even primarily as a sclerosing agent, and/or for use in sclerotherapy, and/or as a tissue irritant, and/or for the treatment of bleeding, in particular bleeding varices, and/or as a surfactant or wetting agent, for example in a product containing another substance such as an active ingredient. Suitably it is not used purely or primarily as an alkylating agent, and/or as an anti-cancer agent. In an embodiment of the invention, the alkyl sulph(on)ate is not used purely or primarily as an antiviral and/or antifungal agent.

In an embodiment, the alkyl sulph(on)ate is used in the absence of an antibiotic, in particular penicillin or a penicillin-type antibiotic such as methicillin or oxacillin, more particularly penicillin. In an embodiment, the alkyl sulph(on)ate is not used in a formulation which is intended and/or adapted and/or suitable for intravenous administration.

Antibacterial activity may be growth inhibitory activity or more preferably biocidal (ie lethal to the relevant organism). It may comprise activity against sessile and/or planktonic bacteria. In the context of this invention, activity against a particular species of micro-organism may be taken to mean activity against at least one, preferably two or more, strains of that species.

Antibacterial activity may be or include the ability to disrupt and/or suppress biofilm formation by the relevant organism; thus, in accordance with the invention, the alkyl sulph(on)ate may be used to treat a condition which is caused, transmitted and/or exacerbated by bacterial bio film formation.

In the present context, the disruption of biofilm formation embraces any negative effect on the ability of a micro-organism to form, maintain or exist in a biofilm, and/or on a biofilm already formed by the organism. Thus, it may involve reducing the amount of a previously formed biofilm, and/or impairing such a biofilm. It may involve killing or inhibiting sessile bacteria within a biofilm.

Suppression of biofilm formation embraces any degree of impairment (including complete prevention) of the ability of a micro-organism to form, or more typically to co-aggregate with, a biofilm. It thus embraces total or partial impairment, including reducing the amount and/or strength of biofilm which the organism is able to form and/or the speed with which it is able to do so. It may involve preventing or reducing the growth or the rate of growth of an existing biofilm formed by the organism.

An antibacterial formulation prepared or used according to the present invention is preferably active as a bactericide, against one or more of the micro-organisms referred to in connection with the first aspect of the invention. In a particularly preferred embodiment of the invention, the formulation is active against one or more bacteria associated with acne, such as P. acnes and in some instances P. granulosum. It is preferably active against propionibacteria which are wholly or partially resistant to one or more antibiotics, for instance those which are in common clinical use. It may for instance be active against macrolide-lincosamide-streptogramin (MLS) resistant and/or macrolide-lincosamide-streptogramin-ketolide (MLSK) resistant bacteria. In particular it may be active against erythromycin-resistant, clindamycin-resistant and/or tetracycline-resistant P. acnes strains of bacteria, the term tetracycline here referring to the class of antibiotics including for example minocycline and doxycycline as well as the specific antibiotic known as tetracycline.

Antibacterial activity may be measured in conventional manner, for instance using the tests described in the examples below. Generally tests for activity involve treating a culture of the relevant bacterium with the candidate antibacterial compound, incubating the treated culture under conditions which would ordinarily support growth of the bacterium, and assessing the level of growth, if any, which can occur in the presence of the candidate compound.

Preferably the alkyl sulph(on)ate has a minimum inhibitory concentration (MIC), at least against propionibacteria, of 125 μg/ml or less, more preferably 62.5 μg/ml or less, yet more preferably 31.25 or 15.6 μg/ml or less, most preferably 10 or 5 or 4 μg/ml or less. Its corresponding minimum biocidal concentration (MBC) is preferably 250 μg/ml or less, more preferably 125 μg/ml or less, yet more preferably 62.5 or 31.25 μg/ml or less, most preferably 15.6 μg/ml or less. Suitably the ratio of its MIC to its MBC is from 0.01 to 1 or from 0.125 to 1, ideally from 0.5 to 1. MIC and MBC values may be measured using conventional assay techniques, for instance as described in the examples below.

Preferably the alkyl sulph(on)ate also exhibits such characteristics in the presence of at least one of, preferably both of, lipid and salt (sodium chloride): these are species which can be present at the surface of the skin and hence performance in this context can be indicative of suitability for use in topical skin treatment formulations.

The concentration of the alkyl sulph(on)ate in a formulation prepared or used according to the invention might suitably be 0.05% w/v or greater, for example 0.1 or 0.5 or 1 or 2% w/v or greater. Its concentration might be up to 20% w/v, preferably up to 15 or 10% w/v, more preferably up to 5 or 4 or 3% w/v, such as from 0.05 to 10% w/v or from 0.05 to 5% w/v or from 0.1 or 0.5 or 1 to 5% w/v. In cases, for instance when the alkyl sulph(on)ate is for use in the treatment of acne, its concentration in the formulation may be for example 1.2 or 1.5 or 2 or 2.5 or 3% w/v or greater.

In accordance with the invention, the alkyl sulph(on)ate is preferably applied topically, to either a living or a non-living surface as appropriate. It is preferably contained in a pharmaceutically acceptable vehicle. Ideally the formulation is suitable for topical application to human or animal, in particular human, skin. It may be suitable for, and/or adapted for, topical administration to the scalp and/or eyes, in particular the eyes. It may in particular be suitable for topical application to such surfaces in or on the human body. A formulation which is ‘suitable for’ topical application may also be adapted for topical application.

A suitable vehicle for a topical formulation will typically be a fluid, which term includes a cream, paste, gel, lotion, foam, ointment, varnish or other viscous or semi-viscous fluid, as well as less viscous fluids such as might be used in drops (for example for use in the eyes). The alkyl sulph(on)ate may be present in the form of a solution or suspension, the term ‘suspension’ including emulsions, micellar systems and other multi-phase dispersions.

The formulation may thus take the form of a lotion, cream, ointment, varnish, foam, paste or gel, or any other physical form known for topical administration. It may comprise a formulation which is, or may be, applied to a carrier such as a sponge, swab, brush, tissue, cloth, wipe, skin patch, dressing or other material designed for application to a tissue surface, to facilitate its topical administration. It may be intended for pharmaceutical (which includes veterinary but is preferably human) use, and/or for cosmetic or other non-medical care purposes (for example, for general hygiene or skin cleansing or for improving the appearance of the eyes).

The vehicle in which the alkyl sulph(on)ate is contained may be any vehicle or mixture of vehicles which is suitable for topical application; the type chosen will depend on the intended mode and site of application. In the context of formulations for topical application to the skin, examples may for instance be found in Williams' Transdermal and Topical Drug Delivery (Pharmaceutical Press, 2003) and other similar reference books. See also Date, A. A. et al, Skin Pharmacol. Physiol., 2006, 19(1): 2-16 for a review of topical drug delivery strategies, and also Skin Delivery Systems, 2006, John J. Wille, Ed., Blackwell Publishing; Textbook of Cosmetic Dermatology, 2004, 3^{rd} edition, Robert Baran, Howard I Maibach, Taylor & Francis; and Skin Care Beyond the Basics, 2001, Mark Lees, Milady.

In the context of formulations for ocular use, examples of suitable vehicles may be found in Ocular Therapeutics and Drug Delivery: A Multidisciplinary Approach (1995, Indra K. Reddy, Ed., CRC Press); and Ophthalmic Drug Delivery Systems (2003, Ashim K. Mitra, Ed., Informa Healthcare).

The alkyl sulph(on)ate may be carried in or on a delivery vehicle which is suitable for targeting or controlling its release at the intended site of administration, for instance to target a desired site and/or time of delivery. Such a vehicle may for instance target the formulation to the skin or hair follicles. It may delay or otherwise control release of the formulation over a particular time period. The alkyl sulph(on)ate may be microencapsulated: suitable encapsulating entities include liposomes, niosomes, aspasomes, microsponges, microemulsions, hydrogels and solid lipid nanoparticles. Particularly suitable liposomes, for topical application to the skin, are those made from stratum corneum lipids, eg ceramides, fatty acids or cholesterol.

In some cases a polar vehicle may be preferred. Where the formulation is intended for use on the skin, the vehicle may be primarily non-aqueous, although in the case of an anti-acne treatment an aqueous vehicle may be used. The vehicle may be surface-active, in particular when it is intended for use in treating surfaces, for instance to cleanse instruments or working areas. It is suitably volatile. In cases the vehicle may be alcohol-based or silicon-based. By way of example, a lotion or gel formulation intended for application to the skin may contain a mixture of water, an alcohol such as ethanol or phenoxyethanol and a glycol such as propylene glycol.

The formulation may contain excipients and other additives known for use in pharmaceutical or veterinary formulations. For example, where it is intended for topical application to the skin, in particular to treat acne, examples of suitable additives include emollients, perfumes, antioxidants, preservatives, stabilisers, gelling agents and surfactants; others may be found in Williams' Transdermal and Topical Drug Delivery (see above). For the treatment of acne, however, it may be preferred for the formulation not to contain an emollient.

Such a formulation may further contain additional active agents such as antimicrobial (in particular antibacterial) agents. For example, it may contain one or more agents selected from anti-acne agents, keratolytics, comedolytics, anti-inflammatories, anti-proliferatives, antibiotics, anti-androgens, sebostatic agents, anti-pruritics, immunomodulators, agents which promote wound healing and mixtures thereof; it may instead or in addition contain one or more agents selected from sunscreens, moisturisers, emollients and mixtures thereof.

An additional antimicrobial agent may be selected from biocides, disinfectants, antiseptics, antibiotics, bacteriophages, enzymes, anti-adhesins, immunoglobulins, antimicrobially active antioxidants and mixtures thereof; it is preferably active as a bactericide, in particular against propionibacteria. It may however be preferred for the alkyl sulph(on)ate to be the only active agent in the formulation, or at least to be the only antimicrobially or antibacterially active agent and/or the only anti-acne active agent.

Where the formulation is for use in the treatment of body odour, it may contain an anti-perspirant such as an aluminium or aluminium-zirconium salt, and/or a deodorising agent. It may be in the form of an aerosol, or of a roll-on or ‘stick’ deodorant of known type, containing appropriate conventional liquid or solid carriers and excipients. It may contain one or more perfumes.

Where the formulation is for use in the treatment of an ocular infection, it may take the form of a cream or ointment, or of eye drops, or of an eye rinse. Such formulations are typically aqueous based.

Where the formulation is for application to a non-living area or surface, for instance as a disinfectant, it may take the form of a solution or suspension of the alkyl sulph(on)ate in an appropriate fluid vehicle such as an alcohol or a water/alcohol mix.

A formulation prepared or used according to the invention may be suitable for, more preferably adapted for, use in an area or on a surface other than living tissue, for instance to treat floors or walls (whether internal or external), work surfaces or instruments, to disinfect contact lenses or to cleanse hair or nails so as to reduce microbe levels. In these cases the excipients, vehicles and/or other additives included with the alkyl sulph(on)ate may be different to those included in a topical skin care formulation, but again may be conventional as known for use in such contexts. A formulation intended for application to a non-living area or surface, for instance as a disinfectant, may contain a higher concentration of the alkyl sulph(on)ate, for example up to 20% w/v or from 1 to 20% w/v.

A formulation prepared or used according to the invention may be incorporated into, and hence applied in the form of, another product such as a cosmetic, a skin or hair care preparation (for example a skin cleanser, toner or moisturiser, or a shampoo, conditioner, styling mousse or gel or hair spray), a deodorant or anti-perspirant, a cleansing preparation (for example a hand wash for use by surgeons prior to treating patients), a pharmaceutical (which includes veterinary) preparation, a cosmeceutical preparation, a toiletry product (for instance a bath or shower additive or a soap), or a laundry or other fabric treatment product. An aspect of the present invention can provide such a product. Moreover the invention can embrace the use of an alkyl sulph(on)ate in the manufacture of such a product for use in the treatment of a bacterial condition.

A formulation prepared or used according to the invention may be marketed with an indication that it has antibacterial and/or anti-acne activity. The marketing of such a formulation may include an activity selected from (a) enclosing the formulation in a container or package that comprises the relevant indication; (b) packaging the formulation with a package insert that comprises the indication; (c) providing the indication in a publication that describes the formulation; and (d) providing the indication in a commercial which is aired for instance on the radio, television or internet. The activity of the formulation may be attributed, in such an indication, at least partly to the presence of the alkyl sulph(on)ate. The invention may involve assessing the activity of the formulation during or after its preparation, for instance against one or more of the pathogens referred to above. It may involve assessing the activity both before and after incorporation of the alkyl sulph(on)ate, for example so as to confirm that the alkyl sulph(on)ate contributes to the activity.

A third aspect of the present invention provides a method for controlling the growth of a propionibacterium, the method comprising applying, to an area or surface which is infected or suspected to be infected or capable of becoming infected with the bacterium, an alkyl sulphate or sulphonate. The alkyl sulph(on)ate may be applied in a formulation as described above, preferably topically. It may in particular be applied to an area or surface which is infected with the bacterium.

‘Controlling the growth’ of a bacterium embraces inhibiting or preventing its growth, whether completely or partially, as well as killing either completely or partially a culture of the bacterium. It also embraces reducing the risk of subsequent growth of the bacterium in or on the area or surface being treated. It may embrace reducing the risk of transmission of the bacterium from the area or surface being treated to another area or surface and/or living body. The method of the invention may thus be used to treat an existing occurrence of the bacterium or to prevent a potential subsequent occurrence. Controlling the growth of a bacterium may also embrace the disruption and/or suppression of bio film formation by the bacterium.

Where the alkyl sulph(on)ate is applied to living human or animal tissue, it may be applied for therapeutic purposes or for non-therapeutic (eg purely cosmetic) purposes. Thus the third aspect of the invention encompasses a method of treatment of a human or animal patient suffering from or at risk of suffering from a condition which is caused by, transmitted by and/or exacerbated by (in particular either caused or transmitted by) propionibacterial activity, the method involving administering to the patient a therapeutically (which term includes prophylactically) effective amount of a formulation containing an alkyl sulphate or alkyl sulphonate. The condition may be any of those referred to in connection with the first and second aspects of the invention. The alkyl sulph(on)ate is suitably administered in an antibacterially effective amount.

Alternatively the alkyl sulph(on)ate may be applied to a non-living area or surface such as in a hospital, dental surgery or food preparation area. For example the method of the third aspect of the invention may be used to treat work surfaces, surgical or other instruments (including for example dental instruments, and toothbrushes or other personal oral health care implements), surgical implants or prostheses, protective clothing such as surgical gloves, contact lenses, clothing, bedding and many other surfaces.

According to a fourth aspect, the invention provides an antibacterial formulation containing an alkyl sulphate or alkyl sulphonate, together with a pharmaceutically acceptable vehicle, the formulation being adapted for topical application. The formulation may contain an anti-perspirant and/or a deodorising agent. The invention also provides, according to a fifth aspect, a product which incorporates a formulation according to the fourth aspect.

According to a sixth aspect, the invention provides a method of increasing the antibacterial and/or anti-acne activity of a formulation, by adding to the formulation an alkyl sulphate or sulphonate. The formulation may for example be a pharmaceutical formulation, typically one which is suitable and/or adapted and/or intended for topical application.

Throughout the description and claims of this specification, the words ‘comprise’ and ‘contain’ and variations of the words, for example ‘comprising’ and ‘comprises’, mean ‘including but not limited to’, and do not exclude other moieties, additives, components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Other features of the invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith. Moreover unless stated otherwise, any feature disclosed herein may be replaced by an alternative feature serving the same or a similar purpose.

The present invention will now be further described with reference to the following non-limiting examples.

### DETAILED DESCRIPTION

Experimental tests were conducted to determine the antibacterial and anti-acne activity of formulations prepared according to the invention.

Test Micro-Organism

The test micro-organism represented the cutaneous propionibacteria. The strain used was Propionibacterium acnes NCTC 737. This is the type strain of the genus; it is fully susceptible to antibiotics. The propionibacteria are clinically significant due to their involvement in acne. This is a very common, complex and multi-factorial skin disease in which P. acnes and other Propionibacterium spp. (for example P. granulosum) play key roles. They are also opportunistic pathogens in compromised hosts. Thus, activity observed against these micro-organisms is expected to be a good predictor of activity against acne.

The propionibacteria were cultured and maintained on Wilkins-Chalgren Anaerobe Medium (agar and broth) at pH 6.0; all cultures were incubated anaerobically at 37° C. for 72 hours.

The following tests were carried out to assess activity against the test organism.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μA of liquid per well. The wells contained liquid culture medium and ranges of decreasing concentrations of the relevant test compound in doubling dilutions (eg 1000, 500, 250, 125 . . . μg/ml, etc, down to 0.49 μg/ml). The culture media were as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, ie the lowest concentration for which the liquid in the well remained clear.

The assays included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Biocidal (Bactericidal) Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target microbial population to recover.

(c) Disc Diffusion Assay (DDA)

This is an internationally recognised standard method for qualitatively assessing the antimicrobial activity of a compound.

A sterile paper disc was impregnated with a sample of the test compound in a suitable solvent and 30 minutes allowed for the solvents to evaporate (where possible). The disc was then placed on an agar plate onto which the test micro-organism had been inoculated. The plate was then incubated under the conditions described above, following which it was examined visually for signs of microbial growth. If the test compound had antimicrobial activity, a circular zone of no growth would be obtained around the disc. The diameter of this zone of ‘inhibition’ was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel may also influence the result.

(d) Supplemented Disc Diffusion Assay

The DDA test may be carried out using an agar gel supplemented with lipid and/or salt to simulate some of the major components present in human skin and to assess whether these substances might reduce the antimicrobial activity observed for the test compound. Performance under these conditions can provide a more reliable indication of activity on topical application. The supplements used in Examples 1 and 2 below were lipid (triolein at 1% v/v) and sodium chloride (100 mM).

### Example 1

### Activity Against Propionibacterium spp—STS

These experiments used P. acnes NCTC 737 as the test organism. MIC, MBC and DDA assays, as described above, were carried out using as the test compound sodium n-tetradecyl sulphate (STS, ex Sigma Aldrich, UK), dissolved in distilled water. All the experiments were conducted in triplicate. For the DDA experiments, 200 μg of the test compound was loaded onto each disc.

The results are shown in Table 1 below; all are collated from a number of experiments.

TABLE 1

| | |
|---|---|
| MIC (μg/ml) | 0.98 |
| MBC (μg/ml) | 0.98 |
| MIC/MBC ratio | 1 |
| DDA (mm) | 29.87 (±1.42) |
| DDA + salt (mm) | 31.62 (±0.47) |
| DDA + lipid (mm) | 33.67 (±0.99) |

It can be seen from Table 1 that the STS is highly active as an antibacterial agent against P. acnes NCTC 737. Furthermore, this activity is increased to some extent in the presence of salt and lipid, which are important constituents of the human skin environment. This indicates the likely activity of the compound as an anti-acne agent, the propionibacteria being implicated in acne.

### Example 2

### Activity Against Propionibacterium spp—Other Alkyl Sulph(on)ates

Example 1 was repeated, using other alkyl sulphates and sulphonates as the test compounds, namely:

(a) lithium dodecyl sulphate, sodium 7-ethyl-2-methyl-4-undecyl sulphate, sodium octyl sulphate, Trizma® (2-amino-2-(hydroxymethyl)-1,3-propanediol, also known as ‘Tris’) dodecyl sulphate, sodium octadecyl sulphate, sodium 1-octane sulphonic acid, sodium 1-butane sulphonate, sodium 1-decane sulphonate, sodium 1-heptane sulphonate, sodium hexane sulphonate, sodium 1-nonanesulphonate, sodium pentane sulphonate, sodium 1-propanesulphate monohydrate, sodium dodecyl sulphate (SDS) and ammonium dodecyl sulphate (ADS), all supplied by Sigma Aldrich, UK;
(b) sodium decyl sulphate, sodium tridecyl sulphate, sodium n-pentyl sulphate and sodium n-hexyl sulphate, all supplied by Acros Organics, UK;
(c) sodium hexadecyl sulphate, sodium 1-dodecanesulphonate, sodium 1-pentadecane sulphonic acid, sodium 1-hexadecane sulphonate, sodium 1-octadecane sulphonic acid, sodium 1-tetradecane sulphonic acid and sodium 1-tridecane sulphonic acid, all supplied by Tokyo Chemical Industry UK Ltd, UK; and
(d) sodium n-nonyl sulphate, triethanolamine lauryl sulphate, sodium n-undecyl sulphate and sodium-n-heptyl sulphate, all supplied by Chemos GmbH, Germany.

All of the test compounds were dissolved in distilled water, with the exception of Trizma® dodecyl sulphate, sodium 1-pentadecane sulphonic acid, sodium 1-hexadecane sulphonate, sodium 1-octadecane sulphonic acid and sodium 1-tetradecane sulphonic acid, which were dissolved in a 50% v/v ethanol:distilled water mixture, and sodium octadecyl sulphate which was dissolved in DMSO.

The MIC/MBC experiments were conducted in triplicate, with the exception of those for SDS and ADS which were conducted in duplicate. The DDAs were performed either in triplicate, in which case standard deviations are quoted in parentheses, or as a single replicate. For the DDA experiments, 200 μg of the test compound was loaded onto each disc. The test compounds, and the results for each, are shown in Tables 2 and 3 below for the sulphates and sulphonates respectively; all are collated from a number of experiments.

TABLE 2
alkyl sulphates of different chain lengths

| | Carbon | | | | | DDA + | DDA + |
|---|---|---|---|---|---|---|---|
| | chain | MIC | MBC | MIC/MBC | DDA | salt | lipid |
| Test compound | length | (μg/ml) | (μg/ml) | ratio | (mm) | (mm) | (mm) |
| Sodium 1-propanesulphate | 3 | >250 | >250 | n/a | 0.00 | 0.00 | 0.00 |
| monohydrate | | | | | (±0.00) | | |
| Sodium n-pentyl sulphate | 5 | >250 | >250 | n/a | 0.00 | 0.00 | 0.00 |
| | | | | | (±0.00) | | |
| Sodium n-hexyl sulphate | 6 | >250 | >250 | n/a | 0.00 | 0.00 | 0.00 |
| | | | | | (±0.00) | | |
| Sodium-n-heptyl sulphate | 7 | >250 | >250 | n/a | 0.00 | 0.00 | 0.00 |
| | | | | | (±0.00) | | |
| Sodium octyl sulphate | 8 | >250 | >250 | n/a | 0.00 | 0.00 | 0.00 |
| | | | | | (±0.00) | | |
| Sodium n-nonyl sulphate | 9 | 250 | 250 | 1 | 0.00 | 0.00 | 0.00 |
| | | | | | (±0.00) | | |
| Sodium decyl sulphate | 10 | 250 | >250 | <1 | 0.00 | 0.00 | 0.00 |
| | | | | | (±0.00) | | |
| Sodium n-undecyl sulphate | 11 | 31.25 | 62.5 | 0.5 | 17.33 | 19.92 | 17.74 |
| | | | | | (±0.65) | | |
| Sodium dodecyl sulphate (SDS) | 12 | 31.25 | 62.5 | 0.5 | 30.70 | 28.22 | 28.22 |
| Ammonium dodecyl sulphate | 12 | 31.25 | 125 | 0.25 | 22.19 | 24.06 | 20.31 |
| (ADS) | | | | | | | |
| Trizma ® dodecyl sulphate | 12 | 31.25 | 62.5 | 0.5 | 0.00 | 0.00 | 0.00 |
| | | | | | (±0.00) | | |
| Lithium dodecyl sulphate | 12 | 15.6 | 15.6 | 1 | 29.99 | 30.51 | 30.19 |
| | | | | | (±0.48) | | |
| Triethanolamine dodecyl | 12 | 3.9 | 15.6 | 0.25 | 30.19 | 28.33 | 25.53 |
| sulphate | | | | | (±0.31) | | |
| Sodium tridecyl sulphate | 13 | 7.8 | 7.8 | 1 | 32.17 | 33.31 | 31.75 |
| | | | | | (±0.65) | | |
| Sodium n-tetradecyl sulphate | 14 | 0.98 | 0.98 | 1 | 29.87 | 31.62 | 33.67 |
| | | | | | (±1.42) | (±0.47) | (±0.99) |
| Sodium 7-ethyl-2-methyl-4- | 14* | 31.25 | 62.5 | 0.5 | 28.53 | 30.82 | 28.64 |
| undecyl sulphate | | | | | (±0.48) | | |
| Sodium hexadecyl sulphate | 16 | 0.98 | 1.95 | 0.5 | 36.32 | 32.37 | 39.84 |
| | | | | | (±1.30) | | |
| Sodium octadecyl sulphate | 18 | 1.95 | 62.5 | 0.03 | 9.41 | 9.30 | 9.92 |
| | | | | | (±0.18) | | |

*The straight part of this branched structure totals 11 carbon atoms.

The data in Table 2 show that a range of alkyl sulphates having carbon chain lengths of 11 or greater are active against P. acnes NCTC 737, and are also therefore suitable candidates for use as anti-acne agents and for use against other conditions in which propionibacteria are implicated. The salt and lipid supplements do not appear to adversely influence antibacterial activity in these tests. Overall, alkyl sulphates with a straight chain length of 14 or more carbon atoms exhibited the greatest level of antimicrobial activity versus P. acnes.

It is of note that the antibacterial activity of free fatty acids, against P. acnes NCTC 737, shows a different pattern with varying chain length. Laurie acid (C12) was found for instance to have an MIC of 15.6 μg/ml and an MBC of 31.25 μg/ml, whereas myristic (C14) and palmitic (C16) acids were found to have both MICs and MBCs of >1000 μg/ml. In other words, P. acnes NCTC 737 appears to be resistant to C14 and C16 fatty acids but surprisingly susceptible to the corresponding alkyl sulphates and (see Table 3) sulphonates.

TABLE 3
alkyl sulphonates of different chain lengths

| | Carbon | | | | | DDA + | DDA + |
|---|---|---|---|---|---|---|---|
| | chain | MIC | MBC | MIC/MBC | DDA | salt | lipid |
| Test compound | length | (μg/ml) | (μg/ml) | ratio | (mm) | (mm) | (mm) |
| Sodium 1-butane sulphonate | 4 | >250 | >250 | n/a | 0.00 | 0.00 | 0.00 |
| | | | | | (±0.00) | | |
| Sodium pentane sulphonate | 5 | >250 | >250 | n/a | 0.00 | 0.00 | 0.00 |
| | | | | | (±0.00) | | |
| Sodium hexane sulphonate | 6 | >250 | >250 | n/a | 0.00 | 0.00 | 0.00 |
| | | | | | (±0.00) | | |
| Sodium 1-heptane sulphonate | 7 | >250 | >250 | n/a | 0.00 | 0.00 | 10.58 |
| | | | | | (±0.00) | | |
| Sodium 1-octane sulphonic acid | 8 | >250 | >250 | n/a | 0.00 | 0.00 | 0.00 |
| | | | | | (±0.00) | | |
| Sodium 1-nonanesulphonate | 9 | >250 | >250 | n/a | 0.00 | 0.00 | 0.00 |
| | | | | | (±0.00) | | |
| Sodium 1-decane sulphonate | 10 | >250 | >250 | n/a | 0.00 | 0.00 | 0.00 |
| | | | | | (±0.00) | | |
| Sodium 1-dodecanesulphonate | 12 | 125 | 250 | 0.5 | 18.05 | 23.04 | 18.37 |
| | | | | | (±1.56) | | |
| Sodium 1-tridecane sulphonic | 13 | 15.6 | 31.25 | 0.5 | 28.64 | 29.26 | 27.39 |
| acid | | | | | (±1.12) | | |
| Sodium 1-tetradecane sulphonic | 14 | 3.9 | 15.6 | 0.25 | 11.16 | 12.40 | 9.61 |
| acid | | | | | (±0.82) | | |
| Sodium 1-pentadecane sulphonic | 15 | 0.98 | 3.9 | 0.25 | 11.78 | 12.09 | 0.00 |
| acid | | | | | (±0.31) | | |
| Sodium 1-hexadecane sulphonate | 16 | 1.95 | 3.9 | 0.5 | 0.00 | 0.00 | 0.00 |
| | | | | | (±0.00) | | |
| Sodium 1-octadecane sulphonic | 18 | 125 | >250 | <0.5 | 0.00 | 0.00 | 0.00 |
| acid | | | | | (±0.00) | | |

Table 3 shows similar results for the alkyl sulphonates. Activity versus P. acnes was detected at chain lengths of C12 and above. The greatest potency was associated with chain lengths between 14 and 16 carbons.

### Example 3

### Topical Anti-Acne Formulations

The results from Examples 1 and 2 show that alkyl sulphates and sulphonates having carbon chain lengths of 11 or greater can be effective against the bacteria associated with acne. This can be of use in preparing antibacterial formulations for use in any context where such bacteria are thought to be involved as possible sources of infection. More specifically, it can be of use in preparing anti-acne formulations, suitably for topical use.

A topical formulation for use in treating acne may for example be prepared by formulating an alkyl sulphate or sulphonate having a carbon chain length of 11 or greater, such as STS, in a suitable fluid vehicle and optionally together with conventional additives. Such vehicles and additives may be for instance as found in Williams' Transdermal and Topical Drug Delivery (see above) and other similar reference books, and/or in Rolland A et al, ‘Site-specific drug delivery to pilosebaceous structures using polymeric microspheres’, Pharm. Res. 1993; 10: 1738-44; Mordon S et al, ‘Site-specific methylene blue delivery to pilosebaceous structures using highly porous nylon microspheres: an experimental evaluation’, Lasers Surg. Med. 2003; 33: 119-25; and Alvarez-Roman R et al, ‘Skin penetration and distribution of polymeric nanoparticles’, J. Controlled Release 2004; 99: 53-62.

The formulation may be prepared and administered using known techniques. It may for example take the form of a cream, lotion, foam, ointment or gel. The concentration of the alkyl sulph(on)ate may be in the ranges described above, dependent on its antibacterial activity and the intended use of the formulation.

1-10. (canceled)

11. A method for controlling the growth of a propionibacterium, the method comprising applying, to a non-living area or surface which is infected or suspected to be infected or capable of becoming infected with the bacterium, an alkyl sulphate or sulphonate having a carbon chain length of 14 or greater.

12. A method of treatment of a human or animal patient suffering from or at risk of suffering from a condition which is caused by, transmitted by and/or exacerbated by propionibacterial activity, the method involving administering to the patient a therapeutically (which term includes prophylactically) effective amount of a formulation containing an alkyl sulphate or sulphonate having a carbon chain length of 14 or greater.

13. A method according to claim 12, wherein the condition is acne.

14. A method according to claim 12, wherein the condition is an eye infection.

15. A method according to claim 12, wherein the alkyl sulph(on)ate is topically applied.

16. A method according to claim 13, wherein the alkyl sulph(on)ate is topically applied.

17. A method according to claim 12, wherein the alkyl sulph(on)ate has a maximum carbon chain length of 18.

18. A method according to claim 13, wherein the alkyl sulph(on)ate has a maximum carbon chain length of 18.

19. A method according to claim 12, wherein the alkyl sulph(on)ate is a C1-4 alkyl sulph(on)ate.

20. A method according to claim 13, wherein the alkyl sulph(on)ate is a C1-4 alkyl sulph(on)ate.

21. A method according to claim 12, wherein the alkyl sulph(on)ate has a linear alkyl chain.

22. A method according to claim 13, wherein the alkyl sulph(on)ate has a linear alkyl chain.

23. A method according to claim 12, wherein the alkyl sulphate or sulphonate is used in a formulation which is in the form of a cream, paste, gel, lotion, foam, ointment, varnish or other viscous or semi-viscous fluid.

24. A method according to claim 13, wherein the alkyl sulphate or sulphonate is used in a formulation which is in the form of a cream, paste, gel, lotion, foam, ointment, varnish or other viscous or semi-viscous fluid.
